(19) United States
(12) Patent Application Publication
(10) Pub. No.: US 2023/0307931 A1
(43) Pub. Date: Sep. 28, 2023

(54) Electronic Device Storing And Powering Assembly

(71) Applicant: Michael King, Canton, MI (US)

(72) Inventor: Michael King, Canton, MI (US)

(21) Appl. No.: 17/701,817

(22) Filed: Mar. 23, 2022

(51) Int. Cl. H02J 7/00; H02J 50/10; A45C 3/02; A45C 15/00; A45C 13/10; A61L 2/10

(52) U.S. Cl. CPC H02J 7/0045; H02J 7/0063; H02J 7/0068; H02J 50/10; A45C 3/02; A45C 15/00; A45C 13/1084; A61L 2/10; A61L 2202/11

(57) ABSTRACT

An electronic device storing and powering assembly for storing, powering, and sanitizing an electronic device includes a container. A lid is hingedly engaged to the container to selectively close an open top of the container. Fasteners, which are engaged to the container and the lid, selectively engage electronic devices. A battery, which is rechargeable, is engaged to and positioned in the container. Ports are operationally engaged to the battery and can engage connectors of power cables of the electronic devices to operationally engage the electronic devices to the battery. The battery thus can power the electronic devices. A power cord is engaged to and is selectively extensible from the container. The power cord is operationally engaged to the battery and the plurality of ports. Engaging the power cord to an outlet charges the battery and provides power to the electronic devices.

### (b) CROSS-REFERENCE TO RELATED APPLICATIONS

Not Applicable

### (c) Statement Regarding Federally Sponsored Research or Development

Not Applicable

### (d) the Names of the Parties to a Joint Research Agreement

Not Applicable

### (e) Incorporation-by-Reference of Material Submitted on a Compact Disc or as a Text File Via the Office Electronic Filing System

Not Applicable

### (f) Statement Regarding Prior Disclosures by the Inventor or Joint Inventor

Not Applicable

### (g) Background of the Invention

### (1) FIELD OF THE INVENTION

The disclosure relates to storing and powering assemblies and more particularly pertains to a new storing and powering assembly for storing, powering, and sanitizing an electronic device. The present invention discloses a storing and powering assembly comprising a container having an integral rechargeable battery, a power strip, and a bulb configured to emit ultraviolet light to kill microbes on electronic devices stowed within the container.

### (2) Description of Related Art Including Information Disclosed Under 37 CFR 1.97 and 1.98

The prior art relates to storing and powering assemblies. Prior art storing and powering assemblies may comprise briefcase like containers having integral power strips for powering and storing electrical devices. What is lacking in the prior art is a storing and powering assembly comprising a container comprising both a rechargeable battery and a power strip having Universal Serial Bus ports and sockets. Further lacking in the prior art is such a storing and powering assembly comprising a bulb configured to emit ultraviolet light to kill microbes on electronic devices stowed within the container.

### (h) Brief Summary of the Invention

An embodiment of the disclosure meets the needs presented above by generally comprising a container, which defines an interior space. A lid is hingedly engaged to the container and is positioned to selectively close an open top of the container. Each of a plurality of fasteners is engaged to a respective one of the container and the lid. The fastener is configured to selectively engage a respective electronic device to removably engage the respective electronic device to the respective one of the container and the lid. A battery, which is rechargeable, is engaged to the container and is positioned in the interior space. A plurality of ports is operationally engaged to the battery. Each port is configured to engage a connector of a power cable of a respective electronic device to operationally engage the respective electronic device to the battery. The battery thus is configured to power the respective electronic device. A power cord is engaged to and is selectively extensible from the container. The power cord is operationally engaged to the battery and the plurality of ports. A plug of the power cord is configured to selectively engage an outlet to operationally engage the battery and the ports to a source of electrical current to both charge the battery and to power the respective electronic device.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front isometric perspective view of an electronic device storing and powering assembly according to an embodiment of the disclosure.

FIG. 2 is a top isometric perspective view of an embodiment of the disclosure.

FIG. 3 is a front view of an embodiment of the disclosure.

FIG. 4 is a rear view of an embodiment of the disclosure.

FIG. 5 is a side view of an embodiment of the disclosure.

FIG. 6 is an in-use view of an embodiment of the disclosure.

### (J) DETAILED DESCRIPTION OF THE INVENTION

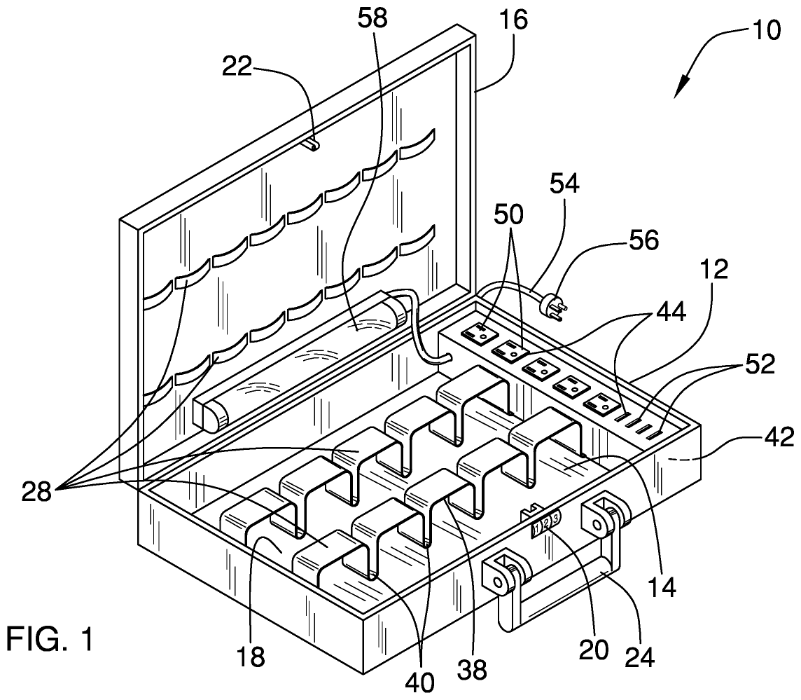

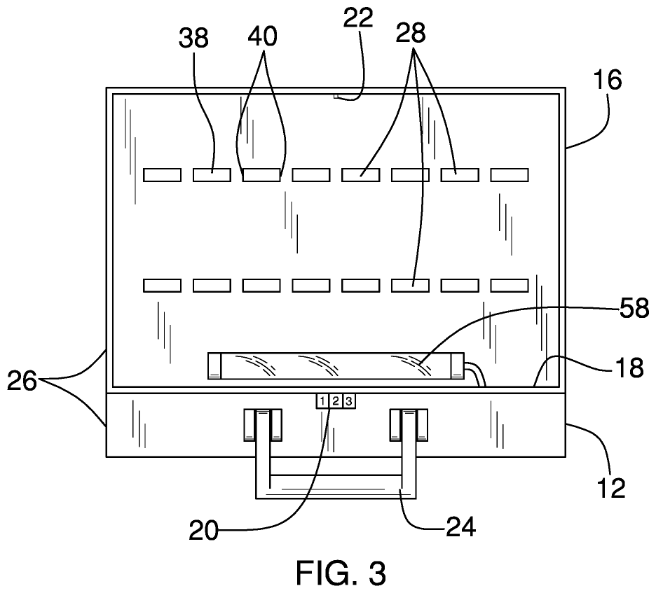

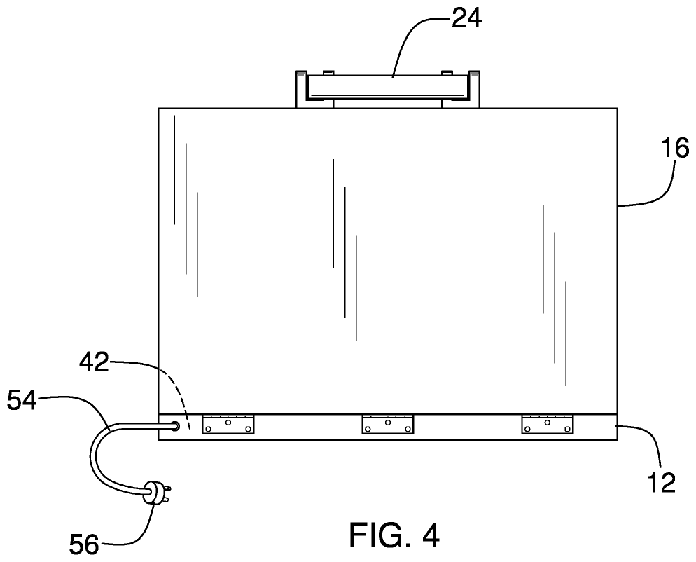

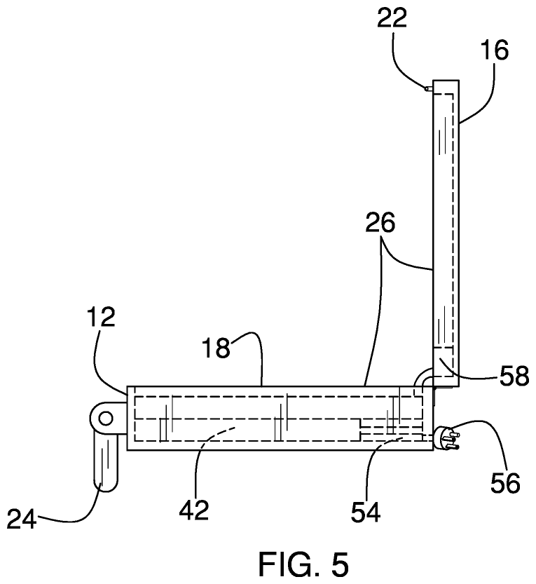

With reference now to the drawings, and in particular to FIGS. 1 through 6 thereof, a new storing and powering assembly embodying the principles and concepts of an embodiment of the disclosure and generally designated by the reference numeral 10 will be described.

As best illustrated in FIGS. 1 through 6, the electronic device storing and powering assembly 10 generally comprises a container 12, which defines an interior space 14. A lid 16 is hingedly engaged to the container 12 and is positioned to selectively close an open top 18 of the container 12. A lock 20 and a latch 22 are engaged to the container 12 and to the lid 16, respectively. The latch 22 is positioned to selectively engage the lock 20 to fixedly position the lid 16 over the open top 18. A handle 24 is hingedly engaged to the container 12 and is configured to be grasped in a hand of a user to lift the container 12. The container 12 and the lid 16 may be configured as a briefcase 26, as shown in FIG. 1, or other containing means, such as, but not limited to, clamshell cases, bags, purses, and the like.

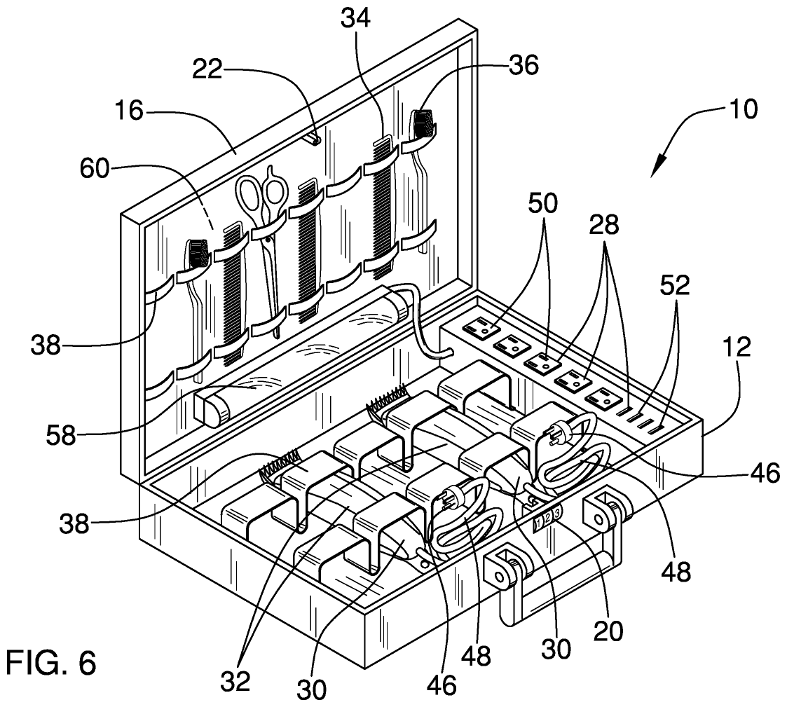

Each of a plurality of fasteners 28 is engaged to a respective one of the container 12 and the lid 16. The fastener 28 is configured to selectively engage a respective electronic device 30 to removably engage the respective electronic device 30 to the respective one of the container 12 and the lid 16. For example, as is shown in FIG. 6, a plurality of hair trimmers 32 is engaged to the container 12 using respective fasteners 28. The fasteners 28 also can be used to attach non-electric devices to the container 12 or to the lid 16, as is demonstrated in FIG. 6 with the attachment of combs 34 and brushes 36 to the lid 16.

Each fastener 28 may comprise an elastic band 38 having opposed ends 40 that are engaged to the respective one of the container 12 and the lid 16. The elastic band 38 is configured to be stretched to allow for insertion of the respective electronic device 30 and to rebound to removably engage the respective electronic device 30 to the respective one of the container 12 and the lid 16. The present invention anticipates the fastener 28 comprising other fastening means, such as, but not limited to, sleeves, depressions, hook and loop straps, clips, and the like.

A battery 42, which is rechargeable, is engaged to the container 12 and is positioned in the interior space 14. A plurality of ports 44 is operationally engaged to the battery 42. Each port 44 is configured to engage a connector 46 of a power cable 48 of a respective electronic device 30 to operationally engage the respective electronic device 30 to the battery 42. The battery 42 thus is configured to power the respective electronic device 30. The plurality of ports 44 may comprise a plurality of sockets 50 and a plurality of Universal Serial Bus ports 52, as shown in FIG. 1. The present invention also anticipates the electronic device storing and powering assembly 10 being used for stowing and recharging cordless electronic devices (not shown) by engagement of charging cords of the cordless electronic devices to the ports 44.

A power cord 54 is engaged to and is selectively extensible from the container 12. The power cord 54 is operationally engaged to the battery 42 and the plurality of ports 44. A plug 56 of the power cord 54 is configured to selectively engage an outlet to operationally engage the battery 42 and the ports 44 to a source of electrical current to both charge the battery 42 and to power the respective electronic device 30.

A bulb 58 is engaged to one of the lid 16 and the container 12. As shown in FIG. 6, the bulb 58 is engaged to the lid 16. The bulb 58 is operationally engaged to the battery 42 and is configured to emit ultraviolet radiation into the interior space 14. The bulb 58 thus is configured to kill microbes upon the respective electronic device 30. The capacity to kill microbes upon electronic devices 30 stowed in the electronic device storing and powering assembly 10 would be of particular benefit to users who are barbers, beauticians, traveling dentists, tattoo artists, and the like.

The present invention also anticipates a primary induction coil (not shown), which is integral to the lid 16 and which is operationally engaged to the battery 42 and the plurality of ports 44. The primary induction coil would be configured to generate a magnetic field. The magnetic field would interact with a secondary induction coil that is integral to a respective electronic device 30, such as a smartphone, to charge a rechargeable battery (not shown) of the respective electronic device 30.

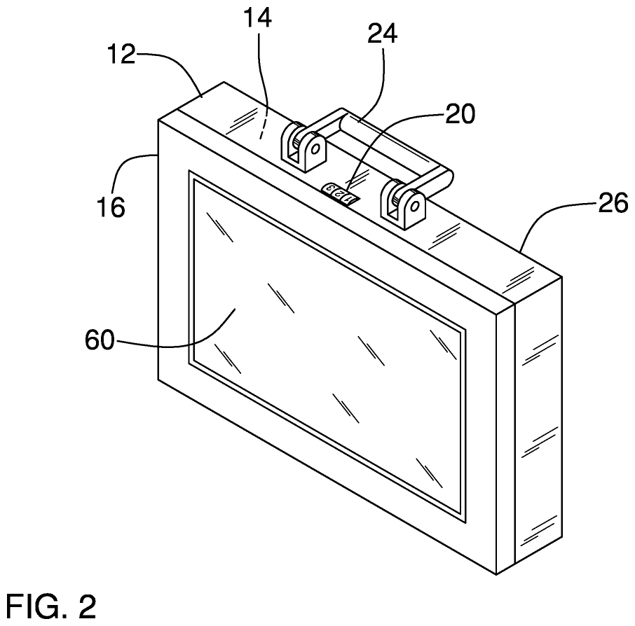

A screen 60 may be engaged to the lid 16, as is shown in FIG. 2. The screen 60 is operationally engaged to the battery 42 and the plurality of ports 44. The screen 60 is configured to display images and text, such as, but not limited to, slides of the user's work.

In use, the electronic devices 30 are fastened to the container 12 using the elastic bands 38. The connectors 46 of power cables 48 of the electronic devices 30 are engaged to the ports 44 to operationally engage the electronic devices 30 to the battery 42. The battery 42 then can power the electronic devices 30 when a source of electrical current is not available. When an outlet is within reach, the power cord 54 can be extended from the container 12 and connected to the outlet to both charge the battery 42 and to provide power to the electronic devices 30. The bulb 58 emits ultraviolet radiation into the interior space 14 to kill microbes upon the electronic devices 30.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An electronic device storing and powering assembly comprising:
a container defining an interior space;
a lid hingedly engaged to the container, such that the lid is positioned for selectively closing an open top of the container;
a plurality of fasteners, each fastener being engaged to a respective one of the container and the lid, such that the fastener is configured for selectively engaging a respective electronic device, wherein the fastener is configured for removably engaging the respective electronic device to the respective one of the container and the lid;
a battery engaged to the container and positioned in the interior space, the battery being rechargeable;
a plurality of ports operationally engaged to the battery, each port being configured for engaging a connector of a power cable of a respective electronic device for operationally engaging the respective electronic device to the battery, wherein the battery is configured for powering the respective electronic device; and
a power cord engaged to and selectively extensible from the container, the power cord being operationally engaged to the battery and the plurality of ports, wherein a plug of the power cord is configured for selectively engaging an outlet for operationally engaging the battery and the ports to a source of electrical current for charging the battery and powering the respective electronic device.

2. The electronic device storing and powering assembly of claim 1, further including a handle hingedly engaged to the container, wherein the handle is configured for grasping in a hand of a user for lifting the container.

3. The electronic device storing and powering assembly of claim 2, wherein the container and the lid are configured as a briefcase.

4. The electronic device storing and powering assembly of claim 1, wherein each fastener comprises an elastic band having opposed ends engaged to the respective one of the container and the lid, wherein the elastic band is configured for stretching for insertion of the respective electronic device and for rebounding for removably engaging the respective electronic device to the respective one of the container and the lid.

5. The electronic device storing and powering assembly of claim 1, further including:
a lock engaged to the container; and
a latch engaged to the lid, such that the latch is positioned for selectively engaging the lock for fixedly positioning the lid over the open top.

6. The electronic device storing and powering assembly of claim 1, wherein the plurality of ports comprises:
a plurality of sockets; and
a plurality of Universal Serial Bus ports.

7. The electronic device storing and powering assembly of claim 1, further including a bulb engaged to one of the lid and the container and being configured for emitting ultraviolet radiation into the interior space, wherein the bulb is configured for killing microbes upon the respective electronic device.

8. The electronic device storing and powering assembly of claim 1, further including a primary induction coil integral to the lid and operationally engaged to the battery and the plurality of ports, wherein the primary induction coil is configured for generating a magnetic field, such that the magnetic field interacts with a secondary induction coil integral to a respective electronic device for charging a rechargeable battery of the respective electronic device.

9. The electronic device storing and powering assembly of claim 1, further including a screen engaged to the lid, the screen being operationally engaged to the battery and the plurality of ports, wherein the screen is configured for displaying images and text.

10. An electronic device storing and powering system comprising:
an electronic device;
a container defining an interior space;
a lid hingedly engaged to the container, such that the lid is positioned for selectively closing an open top of the container;
a plurality of fasteners, each fastener being engaged to a respective one of the container and the lid, a respective fastener being engaged to the electronic device, such that the electronic device is removably engaged to the respective one of the container and the lid;
a battery engaged to the container and positioned in the interior space, the battery being rechargeable;
a plurality of ports operationally engaged to the battery, each port being engaged to a connector of a power cable of the electronic device, such that the electronic device is operationally engaged to the battery for powering the electronic device; and
a power cord engaged to and selectively extensible from the container, the power cord being operationally engaged to the battery and the plurality of ports, wherein a plug of the power cord is configured for selectively engaging an outlet for operationally engaging the battery and the ports to a source of electrical current for charging the battery and powering the electronic device.

11. An electronic device storing and powering assembly comprising:
a container defining an interior space;
a lid hingedly engaged to the container, such that the lid is positioned for selectively closing an open top of the container;
a handle hingedly engaged to the container, wherein the handle is configured for grasping in a hand of a user for lifting the container, the container and the lid being configured as a briefcase;
a plurality of fasteners, each fastener being engaged to a respective one of the container and the lid, such that the fastener is configured for selectively engaging a respective electronic device, wherein the fastener is configured for removably engaging the respective electronic device to the respective one of the container and the lid, each fastener comprising an elastic band having opposed ends engaged to the respective one of the container and the lid, wherein the elastic band is configured for stretching for insertion of the respective electronic device and for rebounding for removably engaging the respective electronic device to the respective one of the container and the lid;
a lock engaged to the container;
a latch engaged to the lid, such that the latch is positioned for selectively engaging the lock for fixedly positioning the lid over the open top;
a battery engaged to the container and positioned in the interior space, the battery being rechargeable;
a plurality of ports operationally engaged to the battery, each port being configured for engaging a connector of a power cable of a respective electronic device for operationally engaging the respective electronic device to the battery, wherein the battery is configured for powering the respective electronic device, the plurality of ports comprising:
a plurality of sockets, and
a plurality of Universal Serial Bus ports;
a power cord engaged to and selectively extensible from the container, the power cord being operationally engaged to the battery and the plurality of ports, wherein a plug of the power cord is configured for selectively engaging an outlet for operationally engaging the battery and the ports to a source of electrical current for charging the battery and powering the respective electronic device;
a bulb engaged to one of the lid and the container and being configured for emitting ultraviolet radiation into the interior space, wherein the bulb is configured for killing microbes upon the respective electronic device;
a primary induction coil integral to the lid and operationally engaged to the battery and the plurality of ports, wherein the primary induction coil is configured for generating a magnetic field, such that the magnetic field interacts with a secondary induction coil integral to a respective electronic device for charging a rechargeable battery of the respective electronic device; and
a screen engaged to the lid, the screen being operationally engaged to the battery and the plurality of ports, wherein the screen is configured for displaying images and text.
